(19) United States
(12) Patent Application Publication
(10) Pub. No.: US 2012/0065533 A1
(43) Pub. Date: Mar. 15, 2012

(54) Positive Airway Pressure System and Method

(76) Inventors: Oscar Carrillo, JR., Morgan Hill, CA (US); Alonzo Charles Aylsworth, Chesterfield, MO (US)

(21) Appl. No.: 13/118,385

(22) Filed: May 28, 2011

Related U.S. Application Data
(60) Provisional application No. 61/349,249, filed on May 28, 2010.

(51) Int. Cl. A61M 16/06; A61M 16/20; A61M 16/00; A61B 5/083

(52) U.S. Cl. 600/532; 128/205.25; 128/205.24; 128/204.23

(57) ABSTRACT

A CPAP system and method which allows the control of released gases from the patient circuit. Coordination of blower speeds and the amount of released gases to improve patient therapy are disclosed. Methods and systems to control patient CO2 retention within the patient mask and to measure patient metabolic function are disclosed.

### CROSS-REFERENCE TO RELATED APPLICATIONS

This non-provisional application claims priority based upon prior U.S. Provisional Patent Application No. 61349249 filed May 28, 2010 in the name of Oscar Carrillo, Jr and Alonzo C. Aylsworth entitled “Positive Airway Pressure System and Method” the disclosure of which is incorporated herein in its entirety by reference.

### STATEMENT REGARDING FEDERALY SPONSORED RESEARCH OR DEVELOPMENT

Not Applicable

### REFERENCE TO SEQUENCY LISTING, A TABLE, OR A COMPUTER PROGRAM LISTING COMPACT DISC APPENDEX

Not Applicable

### FIELD OF THE INVENTION

Embodiments of the present invention is directed to positive airway pressure devices and methods, for example continuous positive airway pressure (CPAP) devices. More particularly, some embodiments of the invention are directed to positive airway pressure devices and methods where the flow and/or pressure to and from the patient are controlled. Additionally, patient metabolism is monitored and system pressure and flow are adjusted in response to metabolic measurements, and patient CO2 levels are controlled and adjusted. Furthermore, embodiments of the present invention are directed to improving the patient CPAP mask.

### BACKGROUND OF THE INVENTION

Sleep disordered breathing is common throughout the population, and some sleep disorder breathing may be attributable to disorders of the respiratory tract. For example, sleep apnea is a situation where a person temporarily stops breathing during sleep. A hypopnea is a period of time where a person's breathing becomes abnormally slow or shallow.

Although hypopneas and apneas may have multiple causes, one trigger for these type events may be full or partial blockages in the upper respiratory tract. In particular, in some patients the pharynx may collapse due to forces of gravity and/or due to forces associated with lower pressure in the upper airway relative to the pressure on the outer wall of the pharynx. A collapse of the pharynx, larynx, upper airway or other soft tissue in the respiratory tract may thus cause the full or partial blockage, which may lead to a hypopnea or apnea event.

One method to counter collapse of the pharynx is the application of constant positive airway pressure to the nostrils, and/or mouth generally, possibly by using a CPAP machine. Application of positive airway pressure may be accomplished in the related art by placing a mask over (and sealing around) the patient's nose and/or mouth, and providing within the mask a pressure communicated to the pharynx, larynx, and upper airway. The pressure within the pharynx, larynx, or upper airway may be greater than the opposing closing forces, thus pneumatically splinting open the airway.

Sleep apnea is defined in the field of sleep disorders as a cessation of breathing during sleep lasting ten seconds or more. Sleep apnea may be characterized as either “central apnea” or “obstructive apnea.” Obstructive apnea is so named because the cessation of breathing is caused by an obstruction in the upper respiratory tract. For example, portions of the soft palate may collapse blocking the airway. In the case of obstructive apnea, the patient may attempt to inhale (i.e. has breathing effort), but the blockage prevents such an inhalation. Central apnea occurs when a sleeping person's central nervous system fails to instruct the diaphragm to retract to draw air into the lungs. Central Sleep Apnea often emerges during the application of CPAP Therapy. Common theory suggests that central apneas may occur when the patient's CO2 levels are reduced or the body's CO2 responsiveness is altered. “Complex Sleep Apnea” is often used to refer to these phenomena. Others have tried adding CO2 inline, increasing dead space between the CPAP with a non-vented mask, and administering Acetazolamide.

Some CPAP machines have the ability to adjust the pressure applied to the patient. In particular, some patients may have difficulty exhaling against the applied pressure, and thus some machines may implement a bi-level CPAP, with a higher pressure applied during inhalation and a lower pressure applied during exhalation. Lowering the pressure reduces the amount of pressure against which the patient must breathe during exhalation. Other CPAP machines continuously adjust the positive airway pressure applied to the mask during inhalation (even if such devices implement a bi-level system), and may be referred to in the related art as “auto titration” devices. With auto titration CPAP devices, as the patient sleeps the positive airway pressure applied is adjusted, cycling between excessive pressures and optimally therapeutic pressures (over pressuring the patient, thus causing an arousal and sleep disruption) and reducing pressure to the point that the patient experiences apneas, hypopneas and/or snoring.

Continuous positive airway pressure (CPAP) machines apply positive airway pressure to a patient's upper airway by way of the nose in an attempt to reduce or alleviate the occurrence of sleep apnea, hypopnea and/or snoring. In order to ensure that a CPAP machine is capable of delivering a prescribed titration pressure, the patient wears a mask that seals either to the patient's face surrounding the nose, the face surrounding the nose and mouth, or to the nostrils of the nose in an attempt to keep the positive air pressure from escaping to atmosphere.

Related art CPAP masks incorporate a vent port, or plurality of vent ports, which provide an intentional leak, the vent leak, to atmosphere allowing the release of exhaled gases. The port system consists of a fixed geometry allowing varying amounts of gases to escape depending on the pressure differential between the interior of the mask and atmospheric pressure. Related art CPAP masks do not maintain a constant leak rate for different pressures.

Related art CPAP systems, especially auto titrating and bi-level systems, may algorithmically misinterpret airflow from the CPAP mask vent port as patient breathing. The misinterpretation may lead to false detection, or measurement, of patient breathing which may lead to improper pressure corrections by related art CPAP systems.

Related art CPAP machines algorithmically determine the presence of a mask leak at the CPAP machine end, and inform the user so that the leak can be addressed. However, these algorithmic mechanisms are relatively insensitive, requiring a substantial mask leak before the algorithm can conclusively determine that a mask leak is present. Moreover, these algorithmic determinations are prone to false indications of a mask leak when in actuality the air escape may be through the mouth, mouth leak. Since mask pressure changes the amount of vent leak, it becomes increasing difficult to assess and quantify the differences between a mask leak and the intentional vent leak, which may lead to false or inaccurate CPAP device response to such a measurement. Related art CPAP and mask devices must therefore make estimations of mask leak and mouth leak since an actual measure is not present.

Accurate measures of patient exhale flow could yield better therapy for patients of certain sleep disorders. Related art CPAP devices and masks do not employ physiological exhale flow quantification and measures, rather related art devices estimate exhale flow quantifications and measures.

Related art CPAP machines and masks do not measure exhaled CO2 or gas density comparisons. These measures could yield better therapy for patients of certain sleep disorders.

The intentional release of gases from related art CPAP masks create a highly undesirable audible noise of escaping gases which often interrupts patient and/or bed partner sleep. Devices common in the art make attempts to dampen the noises generated by the vent leak but patients and bed partners still complain of this noise and of sleep interruption caused by this noise.

The intentional release of gases from related art CPAP masks create a highly undesirable alteration of audible noises that are in synchrony with the patient's breathing. The patient may focus on the breathing noises and lead to an inability to initiate sleep.

The intentional release of gases from related art CPAP masks create airflows that often blow onto the patient and/or bed partners, which often interrupts patient's and/or bed partner's sleep. Devices common in the art make attempts to dampen the airflows from the vent leak but patients and bed partners still complain of the airflow annoyances and of sleep interruption cause by this airflow.

With related art CPAP masks, patients often complain of having a “cold nose”. This often results from the high flow of gases escaping from the vent port in the patient mask. The temperature of the airflow into the patient mask may be lower than the temperature at the patient's nose. This airflow carries the heat away from the patient's nose and exhausts the heat out of the vent port, thus cooling the nose. This situation is more prevalent with patients that require higher pressures for airway stability. Since the vent port is geometrically fixed then the release of airflow gases and patient generated heat is much greater at the higher pressures.

Related art CPAP machines may eliminate patient produced CO2 out of the vent port at too great of a rate. This may lead to CPAP induced central apneas for the patient. This situation is worsened at higher operating pressures since gases escape at a greater rate from the fixed geometry vent port.

Related art CPAP masks maintain the vent port close to the patient airway, either the mouth and/or the nose. The patient side of the mask is at a pressure which is higher than atmosphere. A patient inhale creates a decrease in the pressure within the mask and conversely, an exhale creates an increase in the pressure within the mask. In prior art devices, the resulting inhalation/exhalation pressure swing requires a higher mean pressure value to maintain patient airway patency than if this were not the case. High titration pressures can lead to hypoventilating the patient and/or patient discomfort.

Related art CPAP machines monitor the pressure at the CPAP machine itself. Because of the aforementioned pressure swings at the mask, accurate pressure measurements and control of pressure remotely from the patient mask in the CPAP device is inadequate and imprecise. Unnecessarily high CPAP pressures lower patient compliance to the prescribed therapy.

Some related art CPAP machines attempt to monitor the cardioballistic activity. As the heart beats, the patient's air column is altered and a resultant slight change in pressure and flow is detected. The vent flow reduces the impact of the cardioballistic effect on reaching the CPAP machine. As a result of these factors, related art CPAP machines may be unable to detect cardioballistic data when, in fact, the signal is present. Related art CPAP machines are only able to detect cardioballistic data when breathing is absent.

### BRIEF SUMMARY OF THE INVENTION

A CPAP system and method are disclosed which allows the control of released gases from the patient circuit. Coordination of blower speeds and the amount of released gases to improve patient therapy are disclosed. Methods and systems to control patient CO2 retention within the patient mask and to measure patient metabolic function are disclosed.

### BRIEF DESCRIPTION OF THE SEVERAL VIEWS OF THE DRAWING

FIG. 1a shows a common prior art positive airway pressure device.

FIG. 1b shows a common prior art positive airway pressure device connected to a patient.

FIG. 2a shows a preferred embodiment of the invention.

FIG. 2b shows a preferred embodiment of the invention with flow and pressure sensors on the exhalation side of the flow circuit.

FIG. 3 shows a preferred embodiment of the invention with a variable vent valve on the exhalation side of the flow circuit.

FIG. 4 shows a preferred embodiment of the invention with a CO2 sensor on the exhalation side of the flow circuit.

FIG. 5 shows a preferred embodiment of the invention with a gas density sensor to determine gas composition.

FIG. 6 shows a preferred embodiment of the invention with a valve to allow exhaled air reentry into the inhalation circuit to create a rebreathing circuit.

FIG. 7 shows a preferred embodiment of the invention with multiple valves to allow the control of mixing of inlet and exhaled gases into the inhalation side of the flow circuit.

FIG. 8 shows a preferred embodiment of the invention with the use of a gas density sensor to determine gas composition in lieu of a CO2 or O2 sensor.

FIG. 9 shows a preferred embodiment of the invention with the use of an ultrasonic sensor that can act as a gas density sensor as well as a flow sensor.

### DETAILED DESCRIPTION OF THE INVENTION

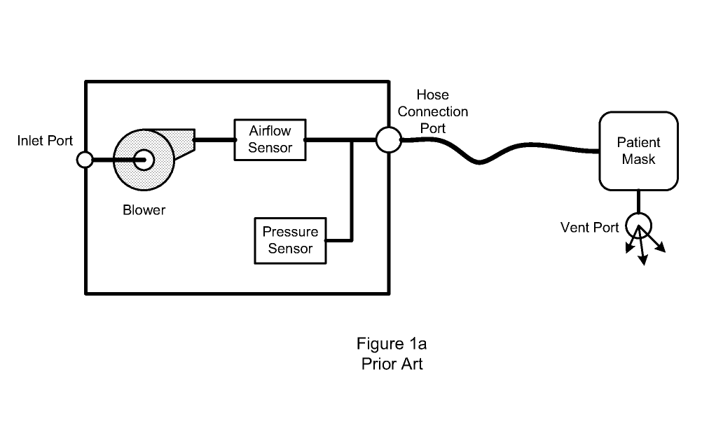

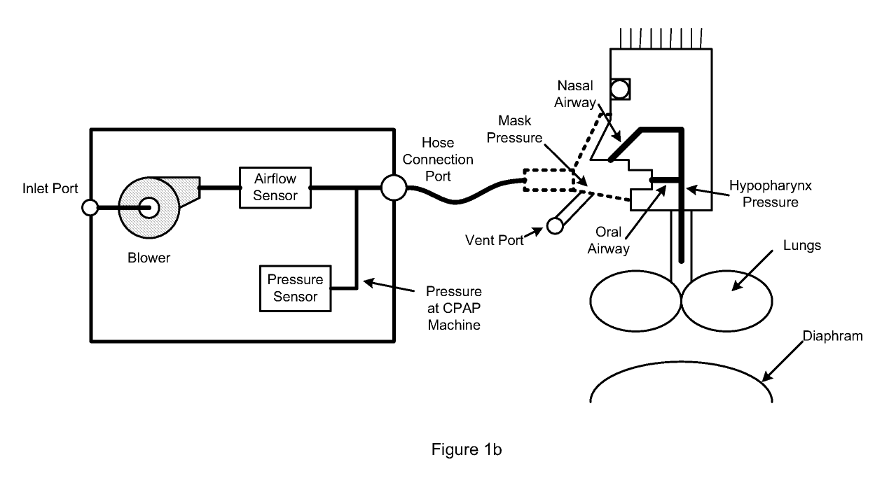

A common prior art device is disclosed in FIG. 1a. Airflow is generated by the blower. Air enters the inlet port and is fed to the blower. Generated airflow is delivered from the blower, through the airflow sensor, and exits the device at the hose connection port, through the patient hose, to the patient mask, and to the patient. A vent port is provided at the mask to expel CO2. The mask typically fits over the patient's nose but may enclose the patient's nose and mouth. Airflow and/or pressures are measured and controlled within the device shown.

Refer to FIG. 1b. Pressure and fluctuations in pressure at any location in the CPAP circuit is determined by the distance from the device and the vent port. In prior art, the vent port is placed in the mask. Rapoport's APSS 2007 Abstract # 0080 abstract shows that therapeutic pressure at the hypopharynx can be drastically different than the mask pressure.

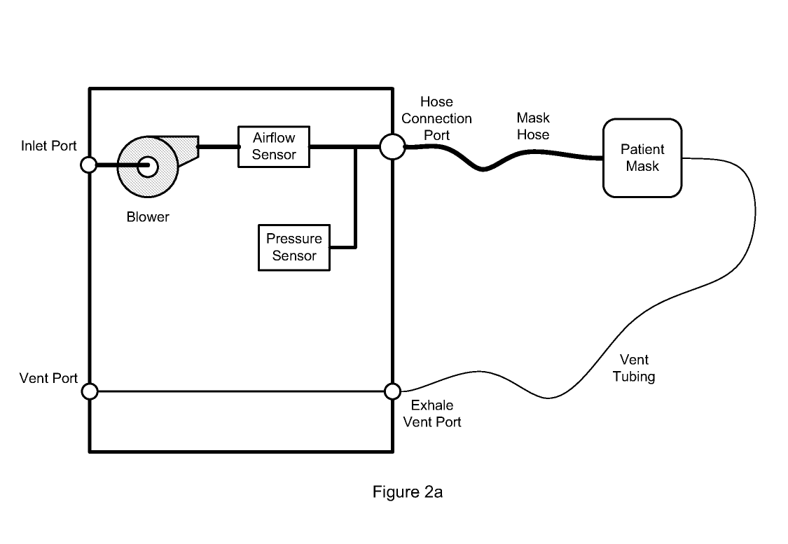

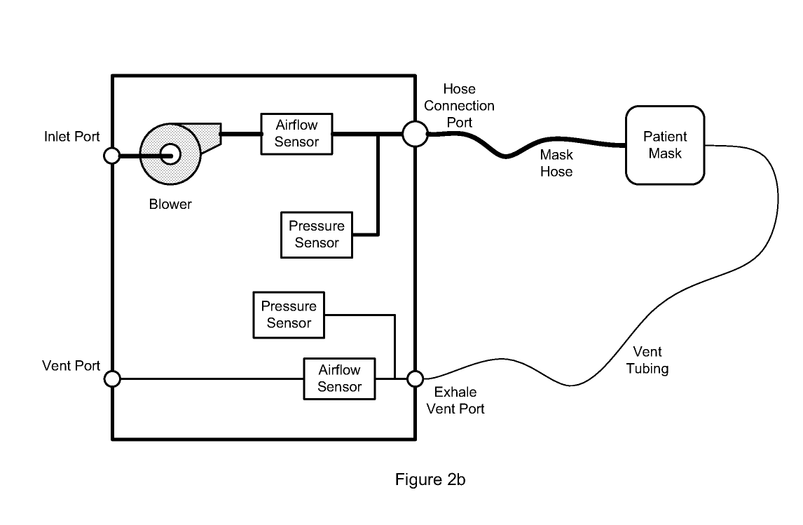

Referring to FIG. 2a, at least one preferred embodiment of the proposed invention, is disclosed herein. As is common in the art, pressure and airflow are generated within the blower in the device. An inlet port allows atmospheric air to enter the inlet of the blower. Airflow under pressure travels through the airflow sensor and through a hose coupled port, as with common art devices. Airflow and pressures are measured within the inventive device as shown. Likewise, at least one patient connected hose is connected from the hose connection port to the patient mask. In this embodiment of our invention, a vent port to atmosphere is not incorporated at the patient mask. However, a vent tube or plurality of vent tubes, are provided herein from the patient mask back to at least the inventive device, said tube, or vent tubing, is connected at the exhale vent port. In this embodiment, the exhale gases exit through the device through the vent port. Those skilled in the art will understand that the release of gases, remote from the patient mask, may take place internal or external of the inventive device.

Placing the vent port remote from the patient mask as disclosed greatly minimizes audible noise of escaping gases, including venting gases and breathing-related fluctuations.

The exhaled gases vented remote of the patient, eliminate the possibility of vented airflow passing over a bed partner or blowing back towards the patient.

Referring to FIG. 2b, the exhale gases enter the exhale vent port, as previously noted. However, analyses of the exhaled gases are herein provided. In this embodiment, the patient's exhaled airflow is monitored with an incorporated airflow sensor. The exhaled gases then exit to atmosphere via a vent port. Monitoring of exhaled gas pressures may also be incorporated in the disclosed pressure sensor. Those skilled in the art will understand that the airflow and pressure sensors do not need to be incorporated into the device. Likewise, the pneumatic sensing of exhaled airflow and pressure may be internal or external to the inventive device.

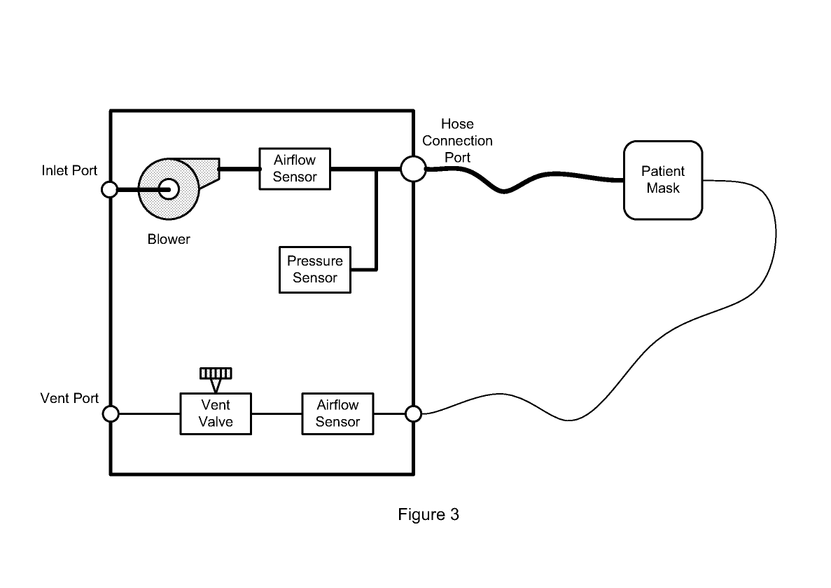

Referring to FIG. 3, in this embodiment exhaled airflow and/or pressures are monitored. However, a vent valve shown is disclosed. Said vent valve may be a device as simple as a fixed orifice, or more complex such as an electronic proportional valve. Those skilled in the art will understand that the vent valve setting may have an impact on the pressure delivered at the mask. For example, for a given value of airflow and pressure, adjusting the vent valve for a smaller leak will create a higher pressure at the patient mask. A lower resistance, larger leak, at the vent valve will create a lower pressure at the patient mask. Thus, disclosed herein is the ability to control mask pressure more accurately, by controlling the leak rate at the vent valve to atmosphere.

It should be noted that the vent tubing from the patient mask which remotely vents to atmosphere may in itself create adequate resistance to airflow to act as an orifice or vent valve described herein. It should also be noted that said vent tubing from the patient mask may be located on the exhalation side as well as on the inhalation side. It is the intention of the invention to measure the flow rate at the vent port, regardless of location of the vent port in the CPAP circuit.

Our invention discloses the ability of the vent port to be geometrically variable, thus regulating the amount of vent flow. The ability to control the exact amount of flow passing thru the CPAP circuit, controls pass-over flow across the patient's nose and airways. Said pass-over airflow is the amount of airflow which is greater than the volume of airflow used by the patient during inhales and exhales. The adjustable vent flow allows us to control the pass-over rate.

In prior art devices, the vent flow rate is a function of primarily the airflow generated by the blower within the common art device and the geometry, or geometric area, of the vent port within the mask. Additionally, the patient's breathing may affect the rate of vent flow in common art devices at the patient's nose. Our invention discloses the ability for the vent valve, preferably in this embodiment an electronically-controlled proportional vent valve, to control the rate of vent flow largely irrespective of the airflow potential which can be generated by the blower and the patient's breathing. Additionally, we disclose herein the ability to control the vent valve via feedback from the airflow sensor in FIG. 3, and/or the pressure sensor connected via the exhalation vent port as disclosed in FIG. 2b.

Those skilled in the art will understand that pneumatic control methods of controlling airflow and/or pressure versus orifice size, or geometric area, at the vent are a feasible means to accomplish the same effect and are within the scope of this invention.

Referring to FIG. 3, our invention incorporates at least one airflow sensor or at least one pressure sensor. In the preferred embodiment, our invention would incorporate at least one airflow sensor and at least one pressure sensor, preferably coupled between the blower and the patient mask. However, those skilled in the art will understand that other pressure and flow attribute sensors may be installed in other locations within the patient airflow system and will yield similar results.

The invention incorporating the vent valve provides the diagnostic capability of detecting patient cardioballistic activity. Accurate cardioballistic data can be used to determine obstructive versus central apneas and to monitor patient heart rate. Our invention enables the ability to monitor heart rate while the patient is breathing. The vent port being remote to the patient mask, and utilizing the vent valve, allows the cardioballistic effect to be more effectively transferred to the circuit airflow, and thus to the sensors within the CPAP device. This allows monitoring of the effect, without the reduction in signal quality that results from related prior art CPAP circuits, whereas the vent port located at the patient mask decreases the signal-to-noise ratio.

Referring to FIG. 3, it should be noted that when the blower is operated at a constant speed, the pressure at the patient mask may be affected by the vent valve. When the vent valve decreases the flow of vent gases then the pressure at the patient mask rises accordingly. When the vent valve allows an increase of vented flow, the pressure at the patient mask decreases accordingly. When the vent valve is set at a fixed orifice size, an increase in blower speed results in an increase in pressure at the patient mask. Conversely a decrease in blower speed results in a decrease in pressure at the patient mask. The variable vent valve capability allows for precise control of the release of gases from the vent port, yet provides for precise control of the pressure at the patient mask via the motor speed of the blower. In at least one scenario, whereas a patient is producing exhaled gases of 6 LPM, the vent valve can be adjusted to allow the release of gases out of the vent port at 6 LPM. Since only adjusting the vent valve affects the patient mask pressure, then it is the intention of this invention to coordinate the release of gases from the vent port and the motor speed to maintain prescribed pressure at the patient mask. For further explanation, adjusting the release of vented gases may require an adjustment to be made to the motor speed to maintain the desired pressure at the patient mask.

Referring to FIG. 3, the arrangement disclosed herein provides an accurate means of detecting and measuring patient circuit leaks, including mask leaks. Preferably by measuring the flow of gases to the patient mask (Inlet Flow) and the flow of gases escaping through the vent port (Outlet Flow) then accurate measurements are possible. The algorithm is (Inlet Flow)−(Outlet Flow)=Instantaneous Leak, and, (Average Inlet Flow)−(Average Outlet Flow)=Average Leak. Monitoring Average Leak vs Instantaneous Leak affords a range of device control means common in the art. However, common art devices do not have the inventive ability to monitor Outlet Flow, and thus, the algorithms used in control of the common art devices are usually based on assumptions and estimations which may not be accurate. Our invention provides the means for accurate control algorithms using Instantaneous Leak and Average Leak. Additionally, accurate reporting of mask leak leads to improved patient therapy. For example, a patient's mask fit may need adjusting due to mask leak. Or, a patient may need a full face mask in lieu of a nasal only delivery mask because of mouth breathing. An accurate measure of the mask leak, or patient circuit leak, allows an accurate determination of the start and end of inhalations and exhalations. This is important in order to control the airflow and pressure attributes versus patient inhalations and exhalations. Referring to FIG. 3, the ability to measure accurate attributes of airflow both prior to the hose connection port and at the exhaled vent port enables accurate measurement of inhalation and exhalation points.

Accurate detection of mask leak is enabled by closing the exhalation vent valve. Measuring flow in the circuit during periods of near-zero, or lack of patient flow, with the vent valve closed to airflow, will allow the ability to only measure leak at the patient which is: mask leak+oral leak.

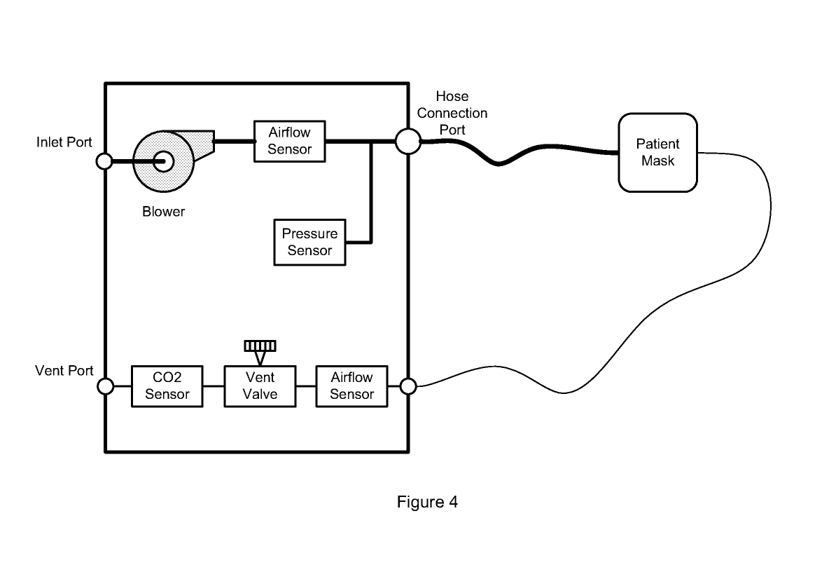

Referring to FIG. 4, our invention includes a CO2 gas sensor disposed within the patient exhaled circuit. As in FIG. 4, the sensor has been placed just prior to the vent port, but those skilled in the art, will understand the CO2 sensor can be placed anywhere along the patient's exhaled circuit. It may be advantageous under certain conditions to have the CO2 sensor coupled directly to the patient mask and not directly coupled to the patient's exhaled circuit.

In this preferred embodiment, the aforementioned CO2 sensor is disposed to provide measurement of patient exhaled gases. CO2, in particular end-tidal CO2, is important because it provides assessment that ventilation is sufficient for metabolic demands.

Measurement of patient exhaled gases, in this case CO2 levels, enables control of patient's ventilation along with the measured CO2 levels. It is the intent of this invention to control the amount of patient ventilation provided primarily by the blower within the device in response to expired CO2 gas measurements. It may be desirable to decrease motor speed in response to a low level of expired CO2 gases. Conversely, it may be desirable to increase motor speed in response to high levels of expired CO2 gases. Additionally, it is the intention of this invention to control said levels of CO2 levels by manipulating the vent valve to increase or decrease CO2 retention in the patient mask. Additionally, it is the intention of this invention to adjust both blower speed and vent valve control to affect CO2 rebreathing within the patient mask.

The vent valve may be closed and pressure adjusted for a period to assist in augmenting patient breathing. Introducing flow into the system is prevented, or at least partially prevented, from exiting the vent port, increasing the likelihood that the flow enters the patient. In the preferred embodiment, the vent valve will be at least partially closed during a patient inspiration and may be at least partially opened during patient exhalation. Coordination of the motor speed may additionally provide assistance in patient breathing during this process and control of CPAP pressure. A period of inspiration and/or expiration may also be treated individually.

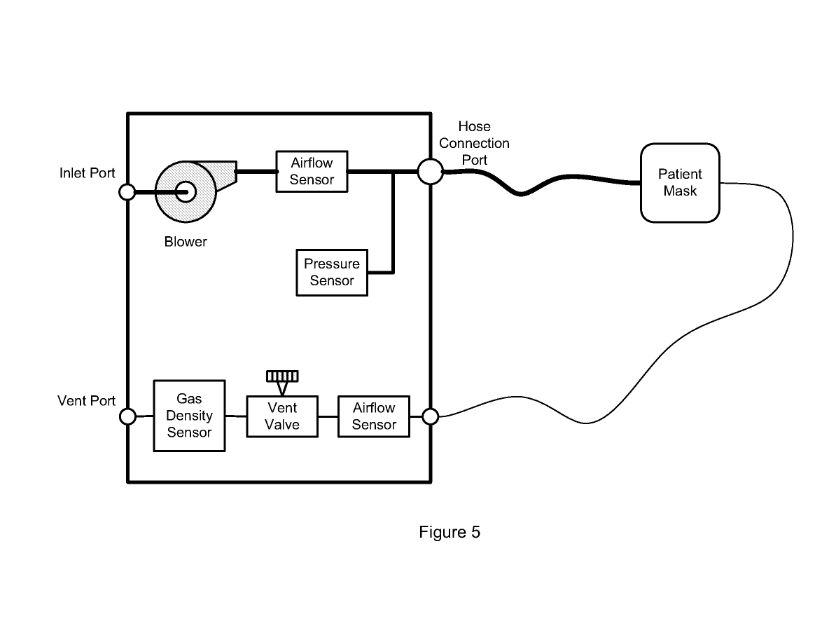

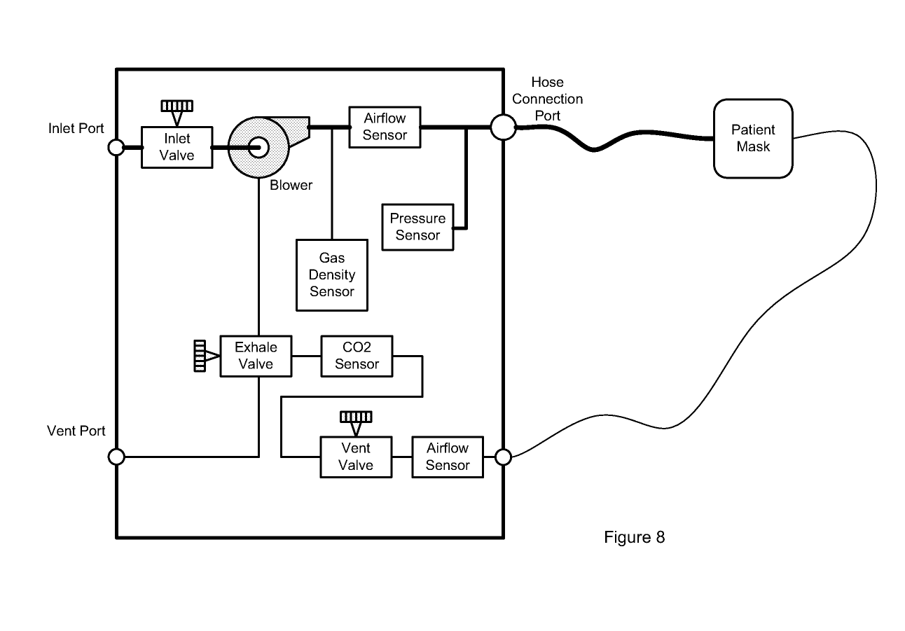

Additionally, it is the intention of this invention, to measure patient circuit leak as previously described herein, and to factor the leak value into the calculations of measured CO2 values to more accurately control the retention of CO2 within the patient's mask. Referring to FIG. 4, it is the intention of this invention to measure the relative humidity of the vented gases preferably at any point along the vented tubing. Whereas said relative levels of humidity measurements may be used in coordination of the operation of the vent valve setting to control the relative humidity of the gases in the patient's mask. Said coordination may also include coordinating the vent valve and/or the blower motor speed to achieve the desired relative humidity. Additionally, whereas said relative humidity sensor is preferably disposed at outlet of the vent valve. Also whereas said relative humidity sensor may be used in conjunction with at least one gas density sensor, refer to FIGS. 5 and 8, to compensate for gas density changes as a result of relative humidity measurements.

Switching the inhalation side with the exhalation side, preferably via a valve, not shown, at the device, will reverse the direction of flow through the CPAP circuit. The reversal of flow through the CPAP circuit will assist in the transfer of the exhaled humidified gases back towards the patient mask. Additionally, this will reduce the amount of condensation in the CPAP circuit.

In our implementation of the CPAP circuit, within the scope of our invention, consisting of inhale side tubing and exhale side tubing, the exhaled humidity and any added humidity will tend to condensate along the path of flow towards the vent port. Reversing the flow will allow the water condensate, due to a lower level of humidity of inlet flow, to return to water vapor and increase humidity for the patient while decreasing water condensate, also known as “rain-out”, in the tubing. It is the intention of the invention to allow the conservation of humidity as well as reducing water condensate in the tubing.

Those skilled in the art will understand that alternate sensors may be used in place of the CO2 sensor as coupled to the patient's exhaled circuit. For example, referring to FIG. 5, a gas density sensor may be coupled to the patient's exhaled circuit. Likewise, an oxygen sensor, may be disposed, or coupled, to the patient's exhaled circuit.

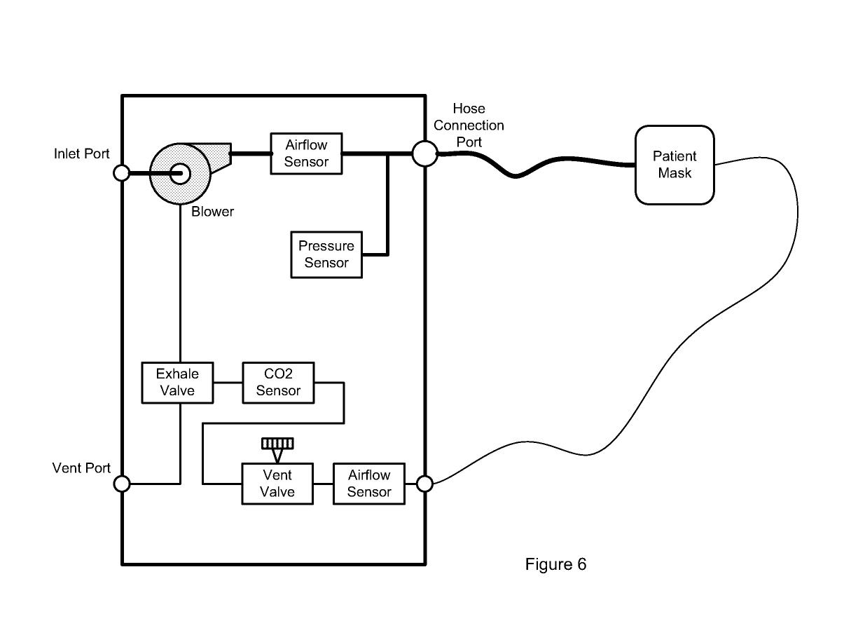

In an alternate configuration, referring to FIG. 6, a gas sensor is coupled along the patient exhaled gas circuit, and the exhaled gases are coupled to an exhale valve. The exhale valve allows at least a portion of the exhaled gases to be coupled to the inlet air system of the device. It is the intention of the invention to allow the control of exhaled gases to be re-introduced into the inlet port of the patient's mask. It may be advantageous in some situations to coordinate the vent valve, the exhale valve, and the blower motor speed to provide the proper amount of CO2 retention in the patient's mask.

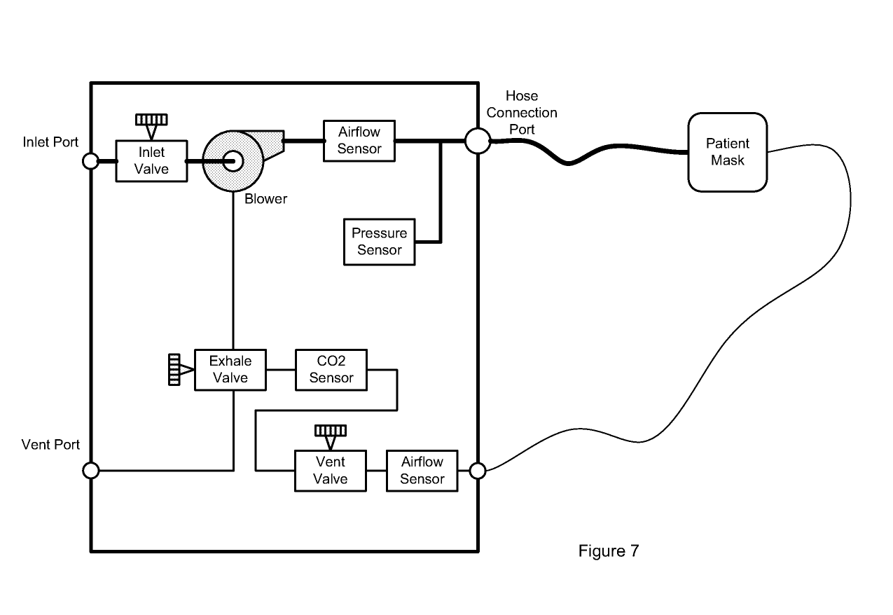

Referring to FIG. 7, an inlet valve is added to the circuit of FIG. 6. Said inlet valve may be used exclusively without the use of aforementioned exhale valve as a means to control the CO2 retention in the patient mask. When the exhale valve is not in the circuit, the exhaled gases are fed directly to the blower. However, in the preferred embodiment, the inclusion of the exhale valve with cooperative control of the inlet valve provides more precise control of the gaseous mixture being fed through the patient's mask. Said gaseous mixture may be monitored by a gas sensor such as a gas density sensor as depicted in FIG. 8. Those skilled in the art will understand that the gas density sensor will be an alternate sensor situation, such as a CO2 sensor and/or O2 sensor. It is the intention of this invention, to have cooperative control between the inhale/exhale valves, the motor speed of the blower, and the vent valve to provide the best gaseous mixture at the optimum airflows and pressures to the patient's mask.

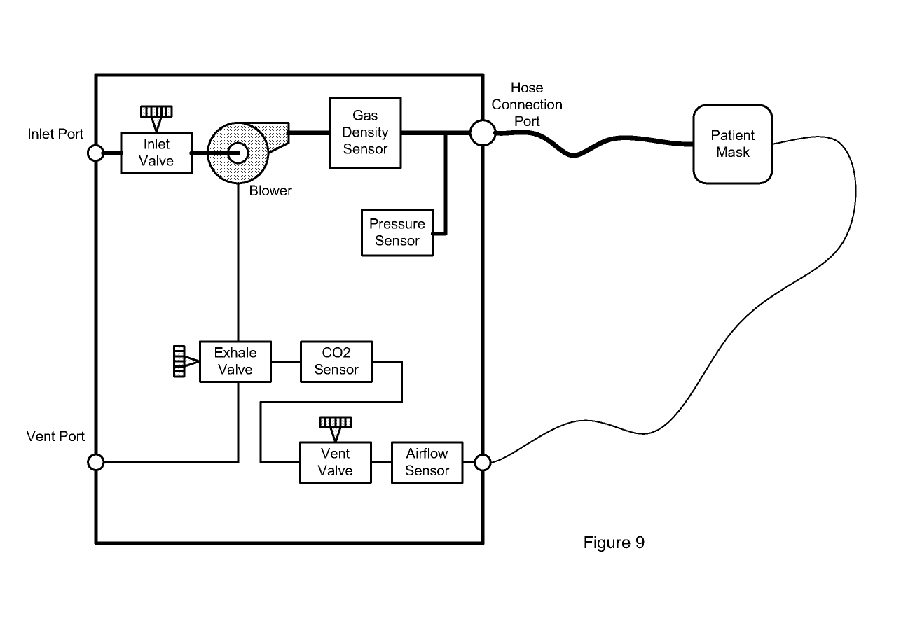

Referring to FIG. 9, the gas density sensor which is coupled between the blower and the hose connection port is an ultrasonic, or speed-of-sound, sensor. Said gas density sensor has the advantage of measuring the gas density or the mixture of the gases being supplied to the patient's mask, but also provides an accurate means of measuring the airflow rate of said gaseous mixture to the patient mask.

It should be noted that said gas density sensor may also be disposed at any point along the patient's inhalation circuit and an additional gas density sensor may be disposed at any point along the patient's exhalation circuit to perform the functions of the previously described exhalation airflow sensors. Additionally, said exhalation gas density sensor may likewise perform the function of measuring exhaled gas density, as previously described herein, and simultaneous airflow measurement.

Said gas density sensor technology is described in the invention of Aylsworth, U.S. Pat. No. 5,060,514.

Combining the measurement of gas composition, specifically CO2 and O2 concentrations, and flow rates, enables the ability to compute the metabolic rate of the patient. Exhaled CO2 concentrations in the exhaled gases reflect cellular CO2 production, and more specifically, CO2 elimination. Exhaled O2 concentrations in the exhaled gases reflect O2 consumption, as the patient extracts O2 from the ambient air for use in cellular processes. It is the intention of this invention to measure the rate of elimination of CO2 (VCO2) and the rate of consumption of O2 (VO2) and the Respiratory Quotient (RQ=VCO2/VO2), in addition to other metabolic parameters.

Combined, VCO2 and VO2, accurately represent the patient's metabolic rate. RQ reflects the composition and utilization of carbohydrates, fats, and proteins as they are converted to energy substrate units.

A patient's metabolic rate can be used to assess the patient's physiological state and status in relation to many other parameters. Metabolic rate is altered with sleep and/or alertness state, circadian rhythm, and infection, and is dependent on lean body mass, body surface area, and body temperature amongst others.

It is the intention of this invention to utilize the metabolic rate to ascertain sleep/wake state, ventilatory sufficiency, and to monitor the health status of the individual as the metabolic rate will change with many physiological states as well as disease processes. Sleep disordered breathing will often change in relation to said physiological states and disease processes. It is the intention of this invention to adjust pressures, flows, and gas mixtures in response to changing physiological states and disease processes, indicated by changes in metabolism.

We claim:

1. A positive airway system whereas the patient ventilation is coupled to at least one vent tube at the patient mask back to the positive airway device.

2. The system as defined in claim 1 further comprising an exhalation port remote from the patient mask for the release of patient exhaled gases to atmosphere.

3. The system as defined in claim 1 further comprising a sensor means to monitor the flow of gases flowing through the vent tube or tubes.

4. The system as defined in claim 1 further comprising a vent valve to control the leak rate to atmosphere.

5. The system as defined in claim 4 further comprising a variable vent valve to vary the rate of exhaled gases.

6. The system as defined in claim 4 further comprising a variable pressure source of therapeutic gas that works in cooperation with the vent valve to control pressure and flow.

7. A method of measuring patient instantaneous mask leak whereas (Inlet Flow)−(Outlet Flow)=Instantaneous Leak

8. A method of measuring patient average mask leak whereas, (Average Inlet Flow)−(Average Outlet Flow)=Average Leak

9. A system of claim 1 to measure mask leak by measuring flow in the circuit during periods of near-zero, or lack of patient flow, with the vent valve of claim 4 closed to airflow.

10. The method of claim 9 to only measure leak at the patient which is: mask leak+oral leak in a nasal-only mask.

11. The system of claim 1 including a CO2 gas sensor disposed within the patient exhaled circuit.

12. The system of claim 11 whereas the CO2 sensor is disposed to provide measurement of patient exhaled gases.

13. The system of claim 11 whereas the CO2 sensor is disposed to measure patient end-tidal CO2.

14. The system of claim 11 whereas the amount of patient ventilation is controlled in response to expired CO2 gas measurements.

15. The system of claim 1 whereas the vent valve may be closed and pressure adjusted for a period to assist in augmenting patient breathing.

16. The system of claim 1 whereas the vent valve will be at least partially closed during a patient inspiration.

17. The system of claim 1 whereas the vent valve will be at least partially opened during patient exhalation.

18. The system of claim 15 where the motor speed is adjustably controlled to provide assistance in patient breathing and control of CPAP pressure.

19. The system of claim 11 whereas the leak valve is used to control the retention of CO2 within the patient's mask.

20. The system of claim 19 whereas the leak value is factored into the calculations of measured CO2 values to more accurately control the retention of CO2 within the patient's mask.

21. The system of claim 1 whereas the relative humidity of the vented gases at any point along the vented tubing is measured.

22. The system of claim 4 whereas the level of relative humidity is controlled by the position of the vent valve.

23. The system of claim 1 whereas an inlet valve is used to control CO2 retention in the patient mask.

24. The system of claim 1 incorporating O2 and CO2 sensors to measure the rate of elimination of CO2 (VCO2) and the rate of consumption of O2 (VO2) and the Respiratory Quotient (RQ=VCO2/VO2) and resulting metabolic parameters.

25. The system of claim 24 to assess the patient's physiological state and status.

26. The system of claim 24 to utilize the metabolic rate to ascertain at least one of the following of a patient: sleep/wake state, ventilatory sufficiency, health status, physiological states, and disease processes.

27. The system of claim 24 to adjust pressures, flows, and gas mixtures in response to changing physiological states and disease processes, indicated by changes in metabolism.
